(19) United States
(12) Patent Application Publication
(10) Pub. No.: US 2026/0284266 A1
(43) Pub. Date: Sep. 24, 2026

(54) FLUIDIC DEVICE FOR WASHING OF LIPOASPIRATE

(71) Applicants: THE REGENTS OF THE UNIVERSITY OF CALIFORNIA, Oakland, CA (US); SAYENZA BIOSCIENCES INC., Irvine, CA (US)

(72) Inventors: Jered Haun, Irvine, CA (US); David Zalazar, Irvine, CA (US); Marzieh Aliaghaei, Rancho Santa Margarita, CA (US); Derek Banyard, Orange, CA (US)

(73) Assignees: THE REGENTS OF THE UNIVERSITY OF CALIFORNIA, Oakland, CA (US); SAYENZA BIOSCIENCES INC., Irvine, CA (US)

(21) Appl. No.: 19/469,807

(22) PCT Filed: Mar. 27, 2024
(86) PCT No.: PCT/US2024/021809

§ 371 Date: Sep. 26, 2025

Related U.S. Application Data
(60) Provisional application No. 63/492,765, filed on Mar. 28, 2023.

(51) Int. Cl. A61L 27/36; B08B 3/08; B08B 3/10

(52) U.S. Cl. CPC A61L 27/3691; A61L 27/3604; B08B 3/08; B08B 3/10

(57) ABSTRACT

A washing device for washing of lipoaspirate includes a cylindrical body having a first inlet and a second inlet located at one end thereof and an outlet located at opposing end of the cylindrical body and one or more spiral ribs disposed along an inner surface of the cylindrical body. A method of washing lipoaspirate includes providing the washing device and loading the washing device with lipoaspirate and a buffer such as saline via the first inlet and second inlet, respectively. Saline is then continuously or semi-continuously pumped into the washing device via the second inlet. Waste effluent is collected via the outlet. After sufficient washing, the washed lipoaspirate is then collected via the outlet. The washed lipoaspirate may be subject to further downstream processing or injected into a subject as a fat graft. Processed lipoaspirate may also be mixed with the washed lipoaspirate for injection in other embodiments.

### RELATED APPLICATION

This Application claims priority to U.S. Provisional Patent Application No. 63/492,765 filed on Mar. 28, 2023 which is hereby incorporated by reference. Priority is claimed pursuant to 35 U.S.C. § 119 and any other applicable statute.

### TECHNICAL FIELD

The technical field generally to fluidic devices for washing of lipoaspirate. In particular, the technical field relates to washing device that has a cylindrical body and one or more spiral ribs disposed on an inner surface thereof. Saline and lipoaspirate is added to the washing device and the device is operated in either a batch or continuous mode to wash, for example, tumescent fluid, blood, and other aqueous phase waste products from the lipoaspirate.

### BACKGROUND

Adipose tissue is used as a filler for fat grafting procedures. Following isolation of the lipoaspirate, it is typically washed with saline to remove contaminants, such as blood and anesthetic drugs. This can be done manually using a flask, and some simple devices have been developed to assist, such as the Revolve™ and PureGraft systems. Manual washing of lipoaspirate is typically performed in several rounds and takes longer than 30 minutes to complete. Moreover, separation efficiency is low, leaving significant blood cell content in the lipoaspirate. Adipose tissue is also an easily accessible source of cells, which are found within the stromal vascular fraction (SVF) and includes a heterogenous mixture of differentiated, stem and progenitor, cells populations. SVF is an exciting candidate for an injectable autologous therapeutic for regenerative applications. Routine processing of lipoaspirate tissue for therapeutic purposes consists of enzymatic dissociation, elimination of adipocytes by centrifugation and collection of SVF and subsequent long-term culture of SVF to isolate Mesenchymal Stem Cells (MSCs), which are like their bone marrow-derived counterparts and can equally show immunosuppressive properties. The first step toward lipoaspirate processing is washing the lipoaspirate to remove blood cells. This step is currently done manually and can result in unreliable results. Additionally, samples contain a high level of oil and blood content variability depending on the patient and harvest site, which would benefit from an automated platform to remove user bias and introduce uniform processing. Also, if this step is performed incorrectly, the remaining blood will affect the purity and functionality of MSCs. It is essential to develop a standard automated method for washing lipoaspirate in MSC collection for clinical applications.

### SUMMARY

In one embodiment, a washing device for washing of lipoaspirate includes a cylindrical body having a first inlet and a second inlet located at one end thereof and an outlet located at opposing end of the cylindrical body. One or more spiral ribs are disposed along an inner surface of the cylindrical body and extend from the end closest to the first and second inlets and extending towards to end with the outlet. The one or more spiral ribs may extend along all or part of the length of the cylindrical body.

In another embodiment, a method of washing lipoaspirate includes providing a washing device having a cylindrical body having a first inlet and a second inlet located at one end thereof and an outlet located at opposing end of the cylindrical body. One or more spiral ribs are disposed along an inner surface of the cylindrical body and extend from the end closest to the first and second inlets and extending towards to end with the outlet. The washing device is loaded with lipoaspirate and a buffer such as saline via the first inlet and second inlet, respectively. Saline is then pumped continuously or semi-continuously into the washing device via the second inlet.

In another embodiment, a method of washing lipoaspirate includes providing a washing device having a cylindrical body having a first inlet and a second inlet located at one end thereof and an outlet located at opposing end of the cylindrical body, wherein one or more spiral ribs are disposed along an inner surface of the cylindrical body and extend from the end of the cylindrical body having the first inlet and the second inlet toward to the opposing end of the cylindrical body. The washing device is loaded with a mixture of lipoaspirate and a buffer such as saline via the first inlet and second inlet, respectively. The mixture of lipoaspirate and saline contained within the washing device is then pumped or recycled out of the outlet and into the first inlet or the second inlet.

A key advantage of the washing device is that lipoaspirate may be washed quickly and thoroughly from blood, drugs, and other waste products. In the dynamic operating mode, complete washing may be done in about 5 minutes or less. The lipoaspirate that is washed with the washing device may be directly used in fat grafting. For example, lipoaspirate may be harvested from a subject, washed using the washing device, collected and then injected as a fat graft back into the same subject. The washing device may also be used in cellular therapeutics by combining the washing device with downstream chemical digestion or mechanical processing to generate cellular therapeutics or supplements to be added to washed lipoaspirate.

### BRIEF DESCRIPTION OF THE DRAWINGS

FIG. 1A schematically illustrates a washing device according to one embodiment.

FIG. 1B illustrates a photograph of the washing device of FIG. 1A.

FIGS. 2A-2C illustrate different embodiments of the washing device. FIG. 2A shows the spiral ribs extending approximately 60% of the length of the cylindrical body of the washing device. FIG. 2B shows the spiral ribs extending approximately 40% of the length of the cylindrical body of the washing device. FIG. 2C shows the spiral ribs extending approximately 20% of the length of the cylindrical body of the washing device.

FIG. 3A illustrates a cross-sectional view of the washing device of FIG. 2A.

FIG. 3B illustrates a cross-sectional view of the washing device of FIG. 2B.

FIG. 3C illustrates a cross-sectional view of the washing device of FIG. 2C.

FIG. 3D illustrates a perspective view of the washing device with the end cap removed showing the spiral ribs inside the cylindrical body of the washing device.

FIG. 4 illustrates another cross-sectional view of the washing device showing the spiral ribs and exemplary dimensions according to one embodiment.

FIG. 5 schematically illustrates the various ways to use the washing device to process lipoaspirate. The washed lipoaspirate may be used directly (1) as a fat graft filler. The washed lipoaspirate may also be subject to further downstream processing (emulsification and micronization and filtration) to generate a stem cell supplement (2). The stem cell supplement may also be combined (3) with washed lipoaspirate for injection as a filler for grafting. The stem cell supplement can improve graft outcomes.

FIGS. 6A and 6B schematically illustrates the workflow of used to operate the washing device in batch mode.

FIGS. 7A and 7B schematically illustrates the workflow of used to operate the washing device in dynamic mode.

FIG. 8A illustrates a bar graph of cell percent viability obtained in lipoaspirate processed using the washer device, the washer device+emulsification (EMD), along with conventional macrofat (obtained with manual washing). Different batch processing times are shown. FW=fat washed or washed with washer device.

FIG. 8B illustrates a bar graph of normalized cell counts obtained in lipoaspirate processed using the washer device, the washer device+emulsification (EMD), along with conventional macrofat (obtained with manual washing). Different batch processing times are shown.

FIG. 8C illustrates a bar graph of population percentages of CD34 expressing cells, endothelial progenitor cells (EPCs), mesenchymal stem cells (MSCs) in lipoaspirate processed using the washer device, the washer device+emulsification (EMD), along with conventional macrofat (obtained with manual washing). Different batch processing conditions/times are shown. FW=fat washed with washer device.

FIG. 9A illustrates a bar graph of cell viability (viability % left axis) in lipoaspirate treated in dynamic mode using a washing device that did not have any spiral ribs. Flow rates and process conditions are shown in the legend.

FIG. 9B illustrates a bar graph of cell count in lipoaspirate treated in dynamic mode using a washing device that did not have any spiral ribs. Flow rates and process conditions are shown in the legend.

FIG. 9C illustrates a bar graph of population percentage for various cell populations (CD34, EPCs, MSCs) obtained by processing lipoaspirate in dynamic mode using a washing device that did not have any spiral ribs.

FIG. 10A illustrates a bar graph of cell viability in lipoaspirate using manual washing, dynamic washing with 0% spiral ribs, 20% spiral ribs, 40% spiral ribs, and 60% spiral ribs.

FIG. 10B illustrates a bar graph of cell count in lipoaspirate using manual washing, dynamic washing with 0% spiral ribs, 20% spiral ribs, 40% spiral ribs, and 60% spiral ribs.

FIG. 10C illustrates a bar graph of population percentage for various cell populations (CD34, EPCs, MSCs) obtained by processing lipoaspirate using manual washing, dynamic washing with 0% spiral ribs, 20% spiral ribs, 40% spiral ribs, and 60% spiral ribs.

FIGS. 11A and 11B illustrate front views of a cap used with the washing device. FIG. 11B illustrates hidden lines showing outlets from the saline inlet.

FIG. 11C is a back view of the cap of FIGS. 11A and 11B.

FIG. 11D is a top view of the cap of FIGS. 11A and 11B.

FIG. 11E is a bottom view of the cap of FIGS. 11A and 11B.

FIG. 12 illustrate another embodiment of the washing device that integrates EMD in a dynamic washing configuration.

FIG. 13A illustrates cell count data obtained using washing device in the configuration of FIG. 12.

FIG. 13B illustrates viability data obtained using washing device in the configuration of FIG. 12.

FIG. 13C illustrates normalized cell population data obtained using washing device in the configuration of FIG. 12.

FIG. 14A illustrate a graph of nucleated cell counts obtained at different wash interval wait times using the setup of FIG. 12.

FIG. 14B illustrate a graph of non-nucleated cell counts obtained at different wash interval wait times using the setup of FIG. 12.

FIG. 14C illustrate a graph of cell viability obtained at different wash interval wait times using the setup of FIG. 12.

FIG. 15 illustrates a graph of normalized cell population data for RBCs, EPCs, and MSCs obtained using washing device in the configuration of FIG. 12.

FIG. 16A illustrates a graph of nucleated cell counts obtained at different total wash volumes using the setup of FIG. 12.

FIG. 16B illustrates a graph of non-nucleated cell counts obtained at different total wash volumes using the setup of FIG. 12.

FIG. 16C illustrates a graph of cell viability obtained at different total wash volumes using the setup of FIG. 12.

FIG. 17 illustrates graph of normalized cell population data for RBCs, EPCs, and MSCs obtained using different washing volumes in the washing device in the configuration of FIG. 12.

FIG. 18A illustrates a comparison of nucleated cell counts obtained using a manual operation with no EMD (MF), the washing device alone with no EMD (PD) as well as syringe-based EMD after manual washing (MF+EMD) and syringe-based EMD after the washing device (PD+EMD).

FIG. 18B illustrates a comparison of non-nucleated cell counts obtained using a manual operation with no EMD (MF), the washing device alone with no EMD (PD) as well as syringe-based EMD after manual washing (MF+EMD) and syringe-based EMD after the washing device (PD+EMD).

FIG. 18C illustrates a comparison of cell viability (%) obtained using a manual operation with no EMD (MF), the washing device alone with no EMD (PD) as well as syringe-based EMD after manual washing (MF+EMD) and syringe-based EMD after the washing device (PD+EMD).

FIG. 18D illustrates a comparison of normalized population of RBCs EPCs, and MSCs obtained macro fat using a manual operation with no EMD (MF), the washing device alone with no EMD (PD) as well as syringe-based EMD after manual washing (MF+EMD) and syringe-based EMD after the washing device (PD+EMD). Cell populations were obtained through flow cytometry.

### DETAILED DESCRIPTION OF ILLUSTRATED EMBODIMENTS

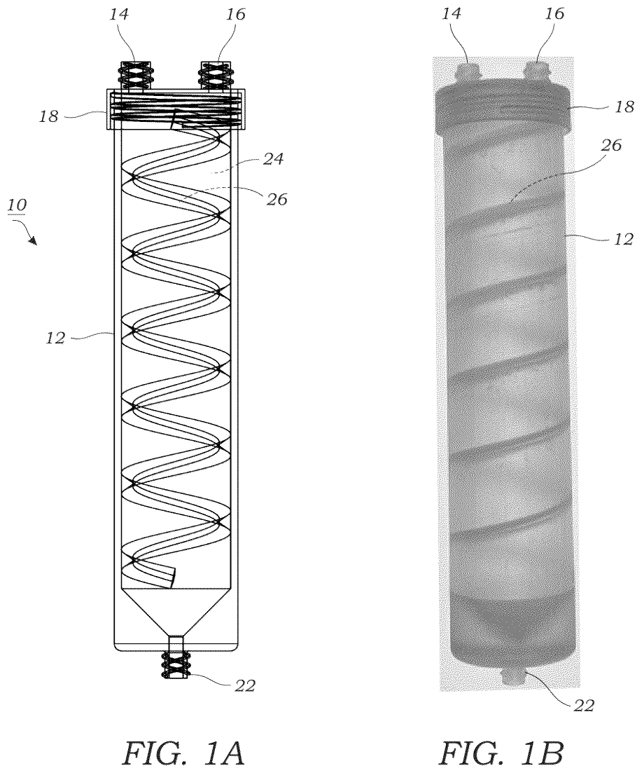

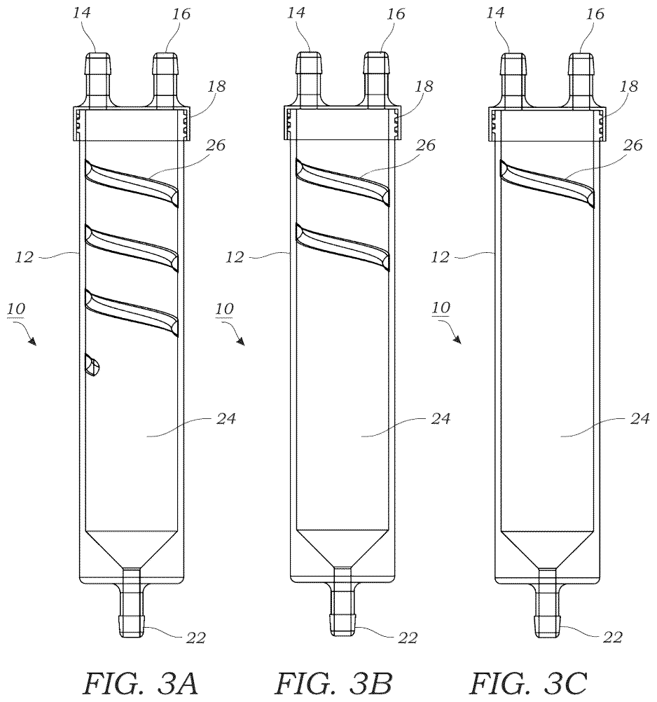

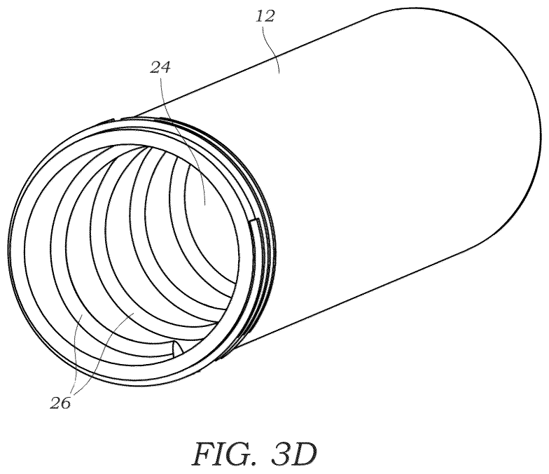

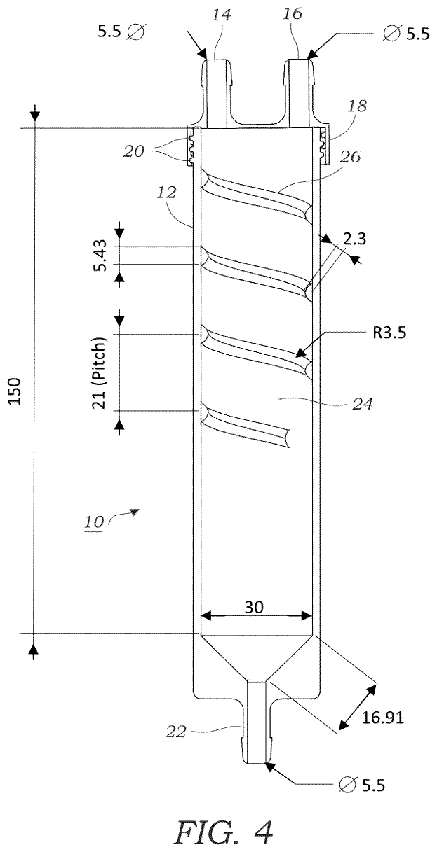

FIGS. 1A, 2A, 2B, 2C, 3A, 3B, 3C schematically illustrates a washing device 10 according to one embodiment. The washing device 10 has a cylindrical body 12 having a first inlet 14 and a second inlet 16 located at one end of the cylindrical body 12. The first inlet 14 and the second inlet 16 may be located on a cap 18 that is secured to the cylindrical body 12 using, for example, threads 20 disposed on the periphery of the cylindrical body 12 (see FIGS. 3A-3C, 4). The first inlet 14 and the second inlet 16, however, may also be integrated directly into the cylindrical body 12. An outlet 22 is located at an opposing end of the cylindrical body 12. The cylindrical body 12 has a lumen or opening 24 that extends between the first and second inlets 14, 16 and the outlet 22. The inner surface of the cylindrical body 12 has one or more spiral ribs 26 that extend partially or fully from the end of the cylindrical body 12 that contains the first and second inlets 14, 16 towards to the outlet 22. FIG. 1B illustrates a photograph of the washing device 10 of FIG. 1A. As seen in FIGS. 1A and 1B, in this particular embodiment, the spiral ribs 26 extend along substantially all (e.g., ~100%) of the inner surface of the cylindrical body 12 between the two opposing ends. As explained below, the spiral ribs 26 may extend along the cylindrical body 12 at different distances in different embodiments.

The cylindrical body 12 may be made from a number of materials including plastics, polymers, or glass. The cylindrical body 12 may manufactured using any number of processes including three-dimensional printing, molding, or the like. The first and second inlets 14, 16 and the outlet 22 may be threaded, barbed, or the like so that that tubing, valves, and the like may be connected to the same. During operation of the washing device 10, the washing device 10 is oriented substantially vertically with the first and second inlets 14, 16 located at the top and the outlet 22 at the bottom. The washing device 10 may be held in a substantially vertical orientation using a stand or rig that secures the cylindrical body 12 vertically.

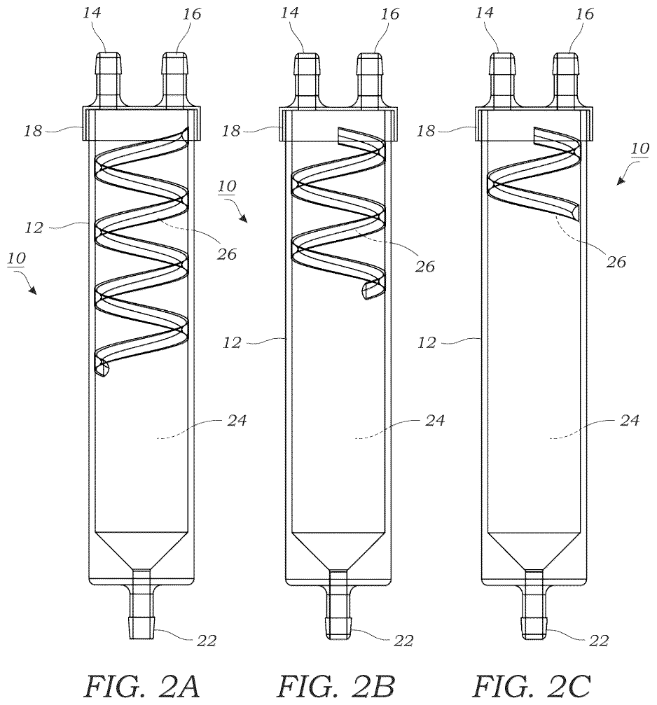

FIGS. 2A-2C illustrates different embodiments of the washing device 10. The embodiments differ by the length or degree of extension of the spiral ribs 26 along the length of the cylindrical body 12. FIG. 2A illustrates an embodiment in which the spiral ribs 26 extend along ~60% of the length of the cylindrical body 12 starting from the end that has the first inlet 14 and the second inlet 16. FIG. 2B illustrates an embodiment in which the spiral ribs 26 extend along ~40% of the length of the cylindrical body 12 starting from the end that has the first inlet 14 and the second inlet 16. FIG. 2C illustrates an embodiment in which the spiral ribs 26 extend along ~60% of the length of the cylindrical body 12 starting from the end that has the first inlet 14 and the second inlet 16.

FIGS. 3A-3C illustrate cross-sectional views of the cylindrical body 12 of the washing devices 10 illustrated in FIGS. 2A-2C. During use of the washing devices 10, the spiral ribs 26 increase the mixing between the buffer solution 102 (e.g., saline) and lipoaspirate 100 that aids in washing out contaminants. By increasing the contact of saline 102 with fat, the spiral ribs 26 aid in removing contaminants such as blood cells or anesthetic drugs from the lipoaspirate 100.

FIG. 4 illustrates a cross-sectional view of a washing device 10 according to one embodiment. In this embodiment, spiral ribs 26 extend along ~60% of the length of the cylindrical body 12 starting from the end that has the first inlet 14 and the second inlet 16. Illustrative dimensions (mm) are shown for the washing device 10. The cylindrical body 12 has a length of 150 mm and an inner diameter of 30 mm. The volume of the washing device 10 is 100 mL in this particular embodiment. It should be appreciated that different volumes are contemplated and the washing device 10 can be scaled up or down as desired. For example, the washing device 10 may have a volume within the range of about 10 mL to about 1000 mL. The spiral ribs 26 extend partially into the interior of the lumen 24 of the cylindrical body 12. In this example, the spiral ribs 26 have a height of around 2.3 mm. Height is measured in the direction orthogonal to the main axis of the cylindrical body 12. The spiral ribs 26 in this example have a width of around 5.43 mm. The pitch between adjacent spiral ribs 26 is 21 mm. The spiral ribs 26 may have a number of profiles or shapes. In the embodiment of FIG. 4, the spiral ribs 26 have generally rounded or smooth profile or shape. The spiral ribs 26 may also have a fin shape or profile in other embodiments as seen, for example, in FIGS. 1A and 1B. The first and second inlets 14, 16 and the outlet 22 have respective diameters of 5.5 mm. It should be appreciated that these dimensions for the washing device 10 are illustrative and other sized and configurations are contemplated.

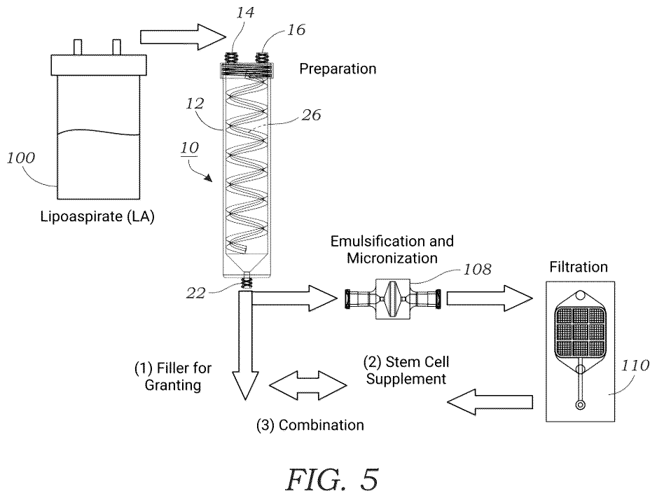

FIG. 5 schematically illustrates the various ways to use the washing device 10 to process or wash lipoaspirate 100. In one aspect, the washed lipoaspirate 100 that is processed with the washing device 10 may be used directly as a fat graft filler. The washed lipoaspirate 100 may also be subject to further downstream processing (e.g., emulsification and micronization and filtration) to generate a stem cell supplement that includes activated Mesenchymal Stem Cells (MSCs), for example. The stem cell supplement may also be combined with washed lipoaspirate 100 for injection as a filler for grafting. The stem cell supplement improves graft outcomes. The use of the washing device 10 allows for automated and consistent washing of lipoaspirate 100 for use in the clinic as an autologous therapeutic. In another embodiment, the washed lipoaspirate 100 is digested with collagenase and cells contained therein are isolated. These cells can then be used directly, or cultured to produce adipose derived stem cells.

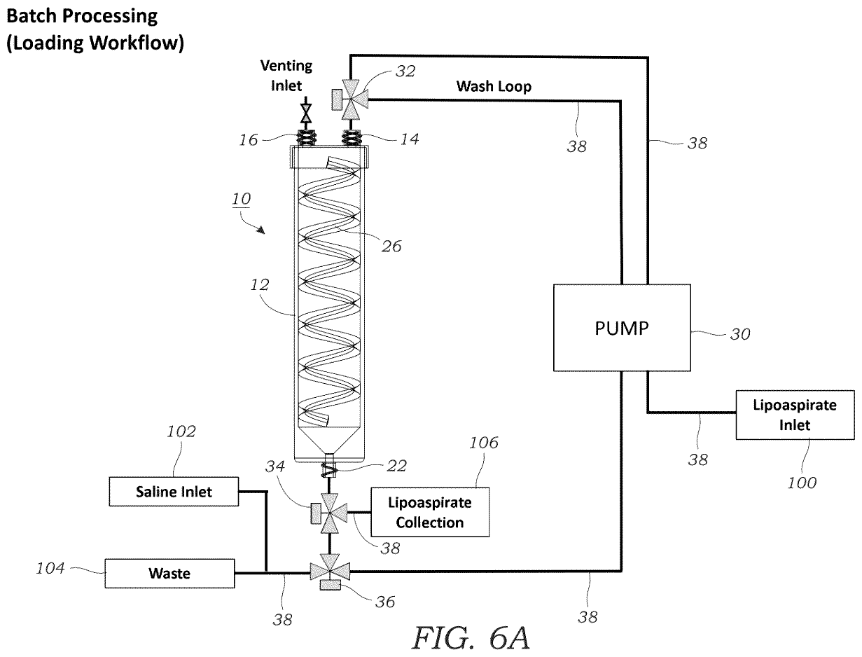

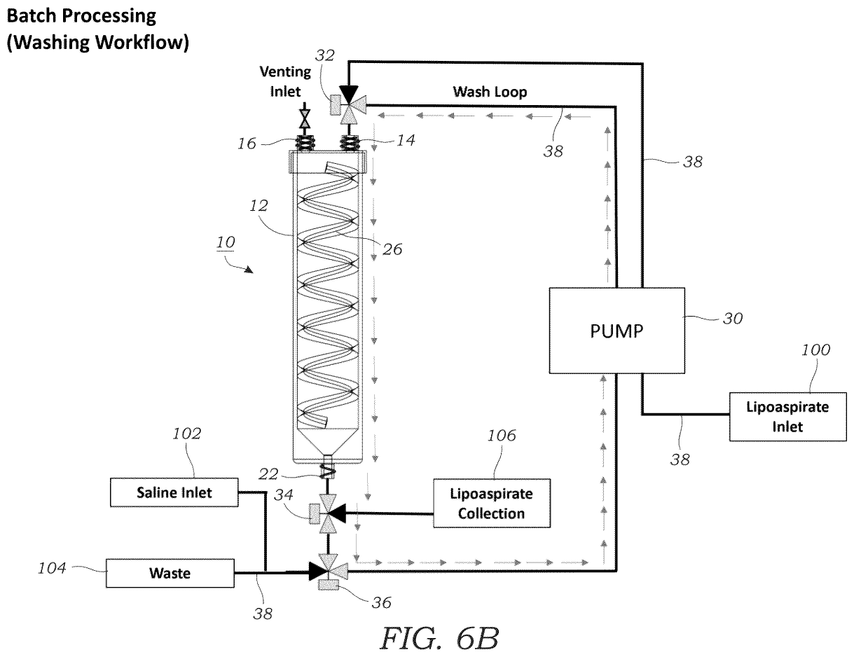

FIGS. 6A and 6B illustrate one exemplary workflow of using the washing device 10. In particular, FIGS. 6A and 6B illustrate a workflow that involves batch processing of lipoaspirate 100. With reference to FIG. 6A, the washing device 10 is loaded with lipoaspirate 100 via pump 30. The same pump 30 is also used to load a buffer such as saline 102 into the washing device 10. The exact proportion of lipoaspirate 100 to saline 102 may vary but may be around 50% (volume basis). The pump 30 may include, for example, a peristaltic pump. The pump 30 may be able to pump multiple flow paths as indicated in FIGS. 6A, 6B. These flows may occur sequentially or simultaneously. A valve 32 may be used to control flow into one of the first inlet 14 or second inlet 16 from either the source of lipoaspirate 100 or the recirculation from the washing device 10 as described herein. Another valve 34 is provided at the outlet 22 which selectively connects to the lipoaspirate collector 106 (e.g., receptacle or container) from the washing device 10. Yet another valve 36 is used to toggle between flowing to waste 104 or recirculation back to the pump 30. Valve 36 is also used to direct flow from saline 102 to the pump 30 during loading of the washing device 10. Tubing or conduit 38 may be used for the flow paths illustrated in FIGS. 6A and 6B.

After the saline 102 and lipoaspirate 100 have been loaded into the washing device 10 having the spiral rib(s) 20, the pump 30 then recirculates the contents through the washing device 10. Specifically, the contents of the washing device 10 leave the outlet 22 and then are pumped via pump 30 back to one of the first inlet 14 or the second inlet 16. A typical flow rate of recirculation is about 5 mL/second. After a period of time, for example between about 1 to about 5 minutes, the pump 30 is stopped and the contents inside the washing device 10 are allowed to settle and undergo phase separation. For some embodiments, for example where the washing device 10 has 100% coverage of the spiral ribs, recirculating the fluid contents for too long (e.g., above 3 minutes) may result in unwanted emulsification. Waste product (infranatant) accumulates at the bottom while the purified lipoaspirate accumulates at the top. The waste can be directed to a waste receptacle 104 via the outlet 22 and valve 36. After the waste has been removed from the washing device 10, the purified lipoaspirate may be collected at valve 34. For example, the purified lipoaspirate may be directed via the valve 34 into a lipoaspirate collector 106 which may include a container, receptacle, or delivery device.

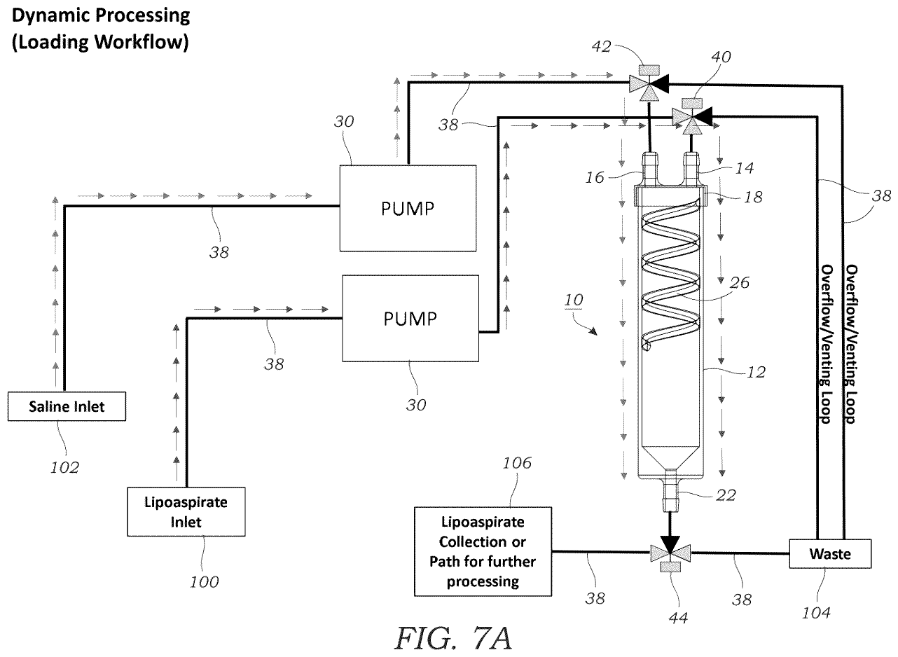

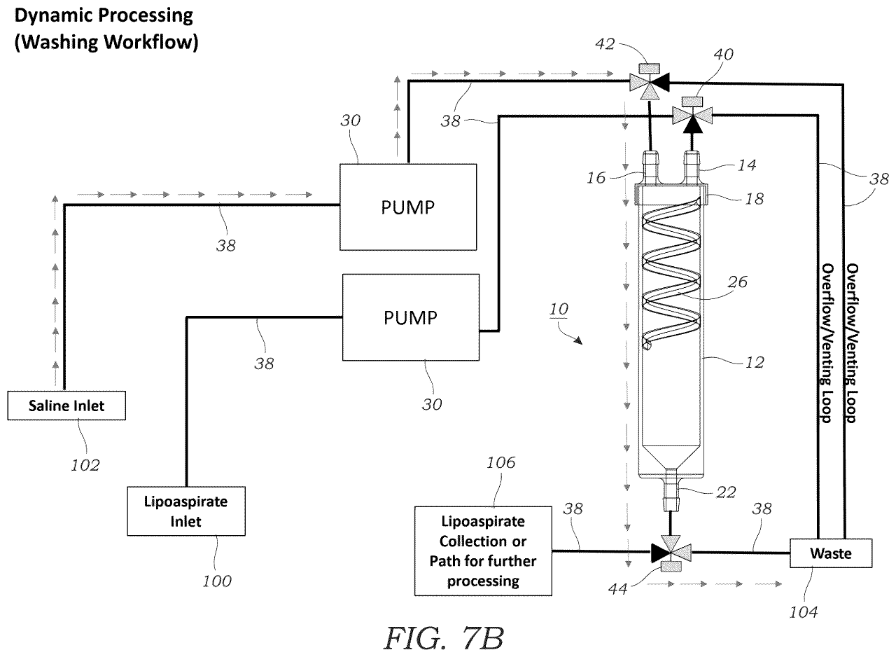

FIGS. 7A and 7B illustrate another exemplary workflow of using the washing device 10. In particular, FIGS. 7A and 7B illustrate a workflow that involves dynamic processing of lipoaspirate 100. With reference to FIG. 7A, the washing device 10 is loaded with lipoaspirate 100 and saline 102 via pumps 30. While two separate pumps 30 are seen in FIGS. 7A and 7B, this could be a single pump capable of pumping two separate flow paths. A source of lipoaspirate 100 is connected to a first inlet 14 of the washing device 10 and a pump 30 such as peristaltic pump is used to pump the lipoaspirate 100 into the washing device 10. The source of lipoaspirate 100 may be a container or bag that contains the lipoaspirate 100. A source of saline 102 (e.g., a container, bag, or reservoir) is connected to second inlet 16 of the washing device 10 and a pump 30 such as a peristaltic pump is used to pump the saline 102 into the washing device 10. Valves 40, 42 interposed between the first and second inlets 14, 16, respectively, are used to direct overflow to waste 104 or used for venting. The exact proportion of lipoaspirate 100 to saline 102 may again vary but may be around 50% (volume basis). The outlet 22 is coupled to a valve 44 that directs the contents of the washing device 10 either to waste receptacle 104 or directs the contents to collection in a lipoaspirate collector 106 or further downstream processing.

With reference to FIG. 7B, the processing of the lipoaspirate 100 begins with no further flow of lipoaspirate 100 into the washing device 10 (e.g., after loading). Valve 40 is closed. Saline 102 is then continuously or semi-continuously pumped in the washing device 10 via the pump 30. Saline is pumped at a range of about 2-2.5 mL/second, although in other embodiments this is increased up to 5 mL/second. The flow rate is chosen to achieve efficient mixing in the portion of the washing device 10 with the spiral ribs 26 and complete separation in the portion of the washing device 10 without spiral ribs 26. The flow rate is a key operating parameter as the saline flow rate through the cylindrical body 12 must be matched to the rate of phase separation, with the spiral ribs 26 providing the mixing element.

Dynamic washing is quicker than batch processing and takes between about 1 and about 3 minutes. Dynamic washing may take place until a pre-determined amount of time has elapsed after starting the washing process. Alternatively, washing may stop after the waste product looks clear. In another embodiment, batch-type recirculation of dynamically washed lipoaspirate 100 may be performed. For example, after a certain volume of saline (e.g., 50 mL) has been added to the washing device 10 during dynamic washing, the contents of the washing device 10 may be recirculated back to the washing device 10 using a recirculation loop and pump 30 like that illustrated in FIGS. 6A and 6B. In this context, a valve 36 may be switched to allow for recirculation back to the washing device 10 to maximize the extraction of waste contents from the lipoaspirate 100. Valve 36 can then be switched back and more saline can be added in a dynamic washing mode until the desired results have been achieved. After stopping the flow of saline 102 with the pump 30, separation is allowed to take place in the washing device 10. Phase separation takes approximately 15 seconds to occur in the washing device 10. Waste product (infranatant) accumulates at the bottom (towards the outlet 22) while the purified lipoaspirate 100 is at the top (towards first and second inlets 14, 16). The waste can be directed to a waste receptacle 104 via the outlet 22 and valve 44. After the waste has been removed from the washing device 10, the washed, purified lipoaspirate 100 may be collected at valve 44 via the lipoaspirate collector 106. The purified lipoaspirate 100 may be used directly, mixed with a stem cell supplement, or sent downstream for further processing (e.g., FIG. 5).

In the dynamic washing embodiment of FIGS. 7A and 7B, after loading of the lipoaspirate 100 into the washing device 10, the pump 30 may pump saline 102 continuously until the end of processing. In another embodiment, the pump 30 may pump semi-continuously. That is to say, the pump 30 may pump for an elapsed period of time and stop for a few seconds at which point the pump 30 is restarted. For example, in one embodiment the saline 102 is pumped at 5 mL/sec for 5 seconds. The pump 30 then remains off for 10 seconds and restarts at a lower flow rate of 2.5 mL/sec for 10 seconds, followed by 10 seconds in the off state. This may be repeated for several cycles (e.g., 3 cycles). This produces a total wash volume of 150 mL and a total wash time of 1 minute 45 seconds. Three cycles result in an elapsed time of about 6 minutes. This is much quicker than the ≥30 minutes it takes for manual washing.

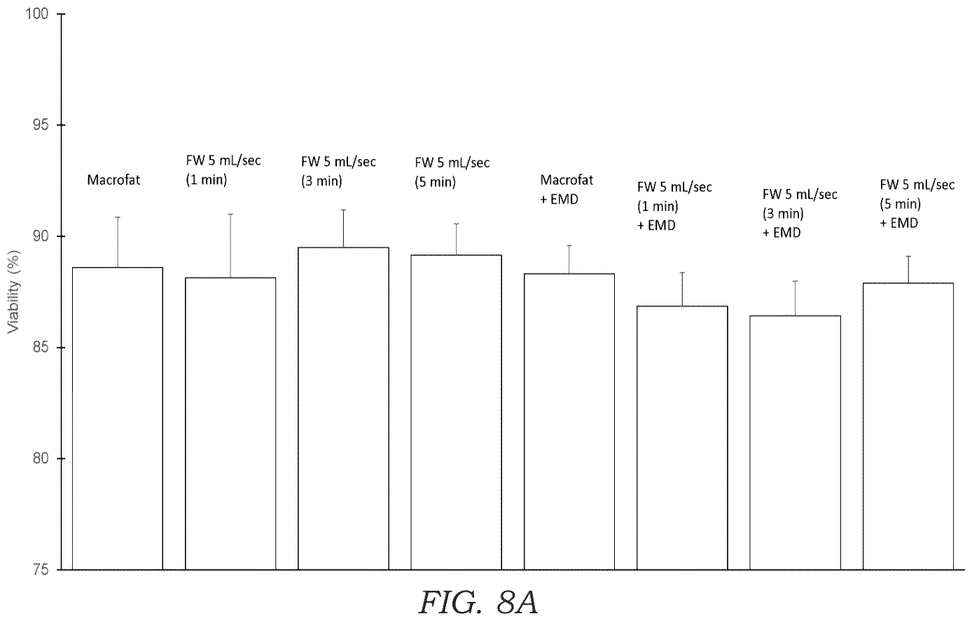

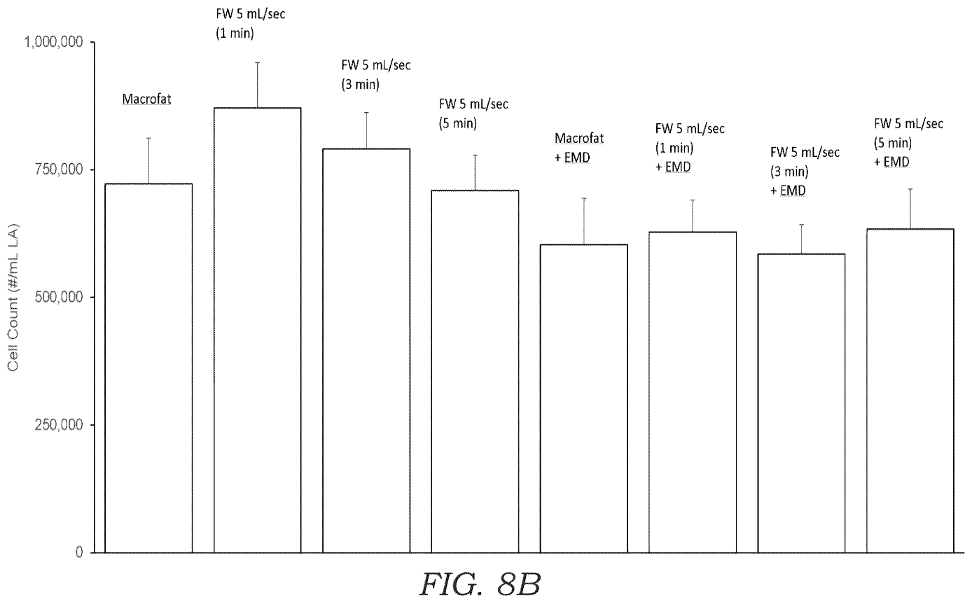

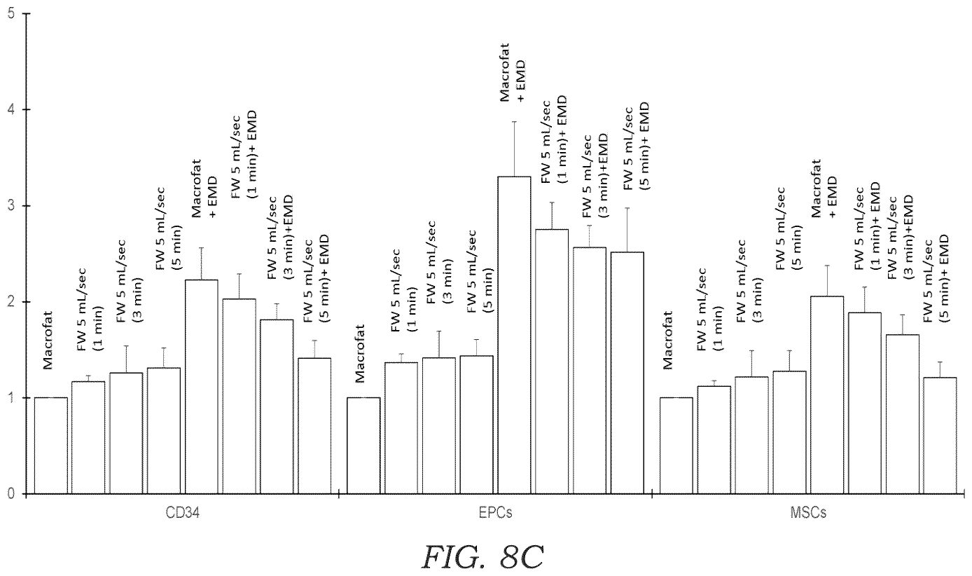

FIGS. 8A and 8B illustrate results (viability and cell count) of batch processing using the washing device 10. Washing with the washing device 10 for 1 minute had cell counts higher than macrofat (FIG. 8B). Unprocessed lipoaspirate 100 is indicated as macrofat. In addition, cell percent viability was higher for all fat washed samples as compared to macrofat (FIG. 8A). Here, the washing device 10 contains a spiral rib 26 along 100% of the cylindrical body 12. Flow rates were 5 mL/sec and results obtained after 1 min, 3 min, and 5 min. In addition to testing the washed lipoaspirate using just the washing device 10, the washed lipoaspirate was subject to downstream processing using an Emulsification and Micronization Device (EMD) 108. The EMD 108 featured two 1.5 mm diameter constrictions separated by an abrupt expansion (FIG. 5). The constrictions generate shear forces to break down connective tissue into smaller units, while the expansion achieves turbulent mixing needed to emulsify the fatty oil layer. While not tested here, an optional filtration device 110 (FIG. 5) may be used to remove large adipose tissue fragments, as well as to further increase cell recovery via tissue dissociation. Additional details regarding the EMD 108 and filtration device 110 may be found in U.S. Patent Application Publication No. 2022/0249761, which is incorporated herein by reference. In one embodiment, the washing device 10 is integrated with the EMD 108 and filtration device 110 to create a closed-loop fluidic system to maximize processing efficiency and the enable automation. This platform can be used to produce both graft filler and mechanically processed, MSC-laden supplement for optimal graft healing and long-term retention. Filler and supplements will be evaluated using in vitro assays.

FIG. 8C illustrates the population % (normalized to macrofat) of batch processed lipoaspirate 100 using the same conditions described above with respect to FIGS. 8A and 8B. As seen in FIG. 8C, increased percentages are seen for cells positive for CD34, EPCs, and MSCs for fat washed samples. Fat washed samples along with EMD 108 resulted in the highest degree of enrichment.

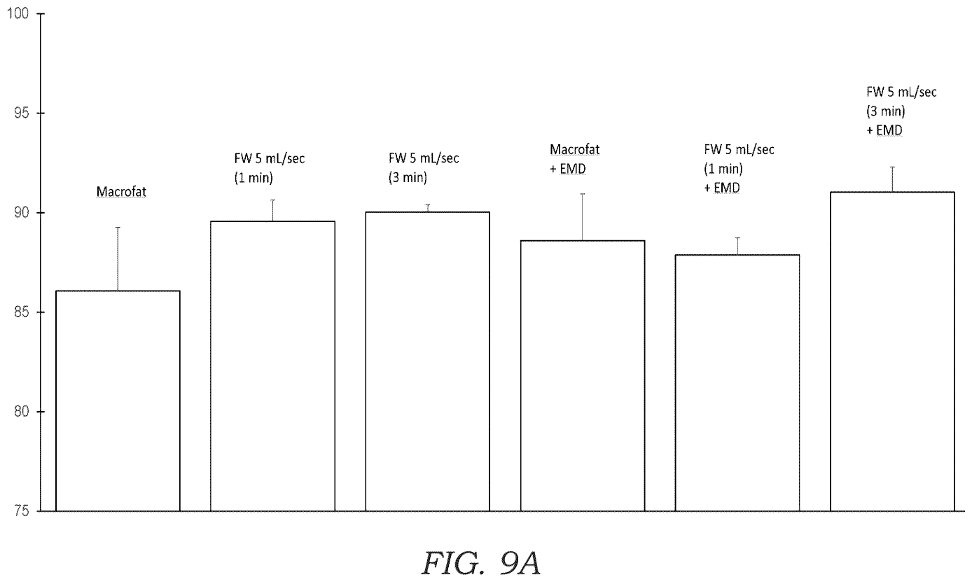

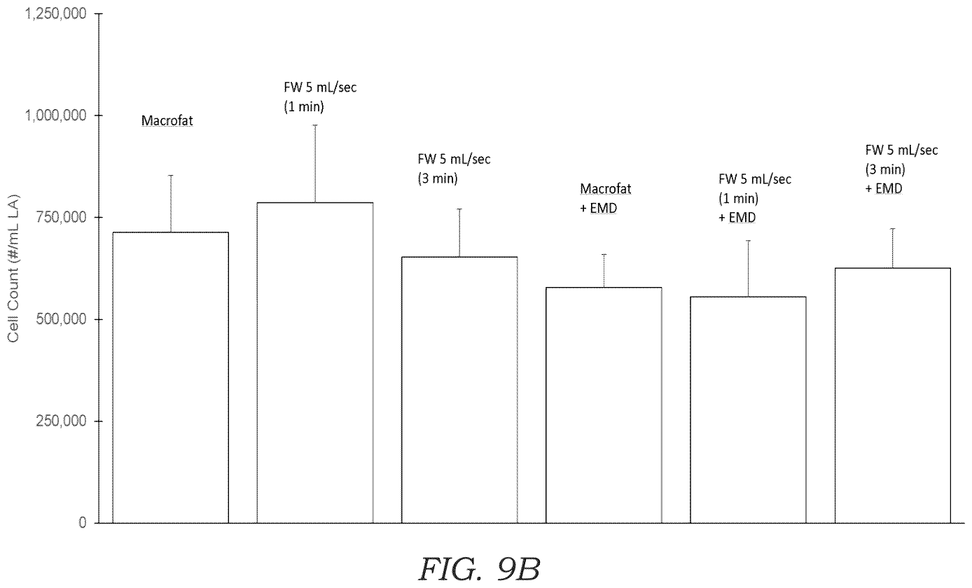

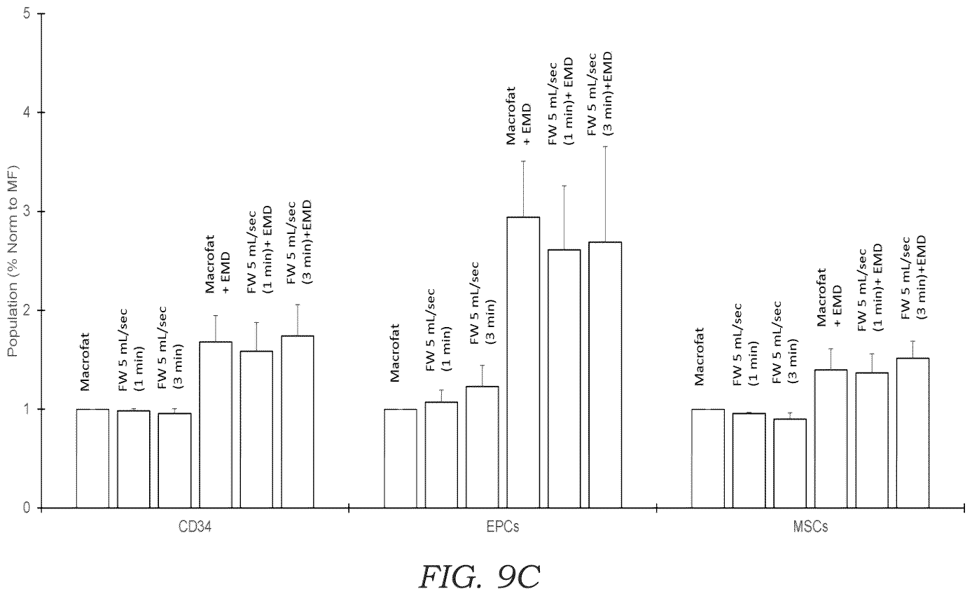

FIGS. 9A and 9B illustrate bar graphs of cell viability and cell count, respectively, in lipoaspirate 100 treated in batch mode using a washing device 10 that did not have any spiral ribs 26. FIG. 9C illustrates bar graphs of various cell populations (CD34, EPCs, MSCs) obtained by processing lipoaspirate 100 in batch mode using a washing device 10 that did not have any spiral ribs 26.

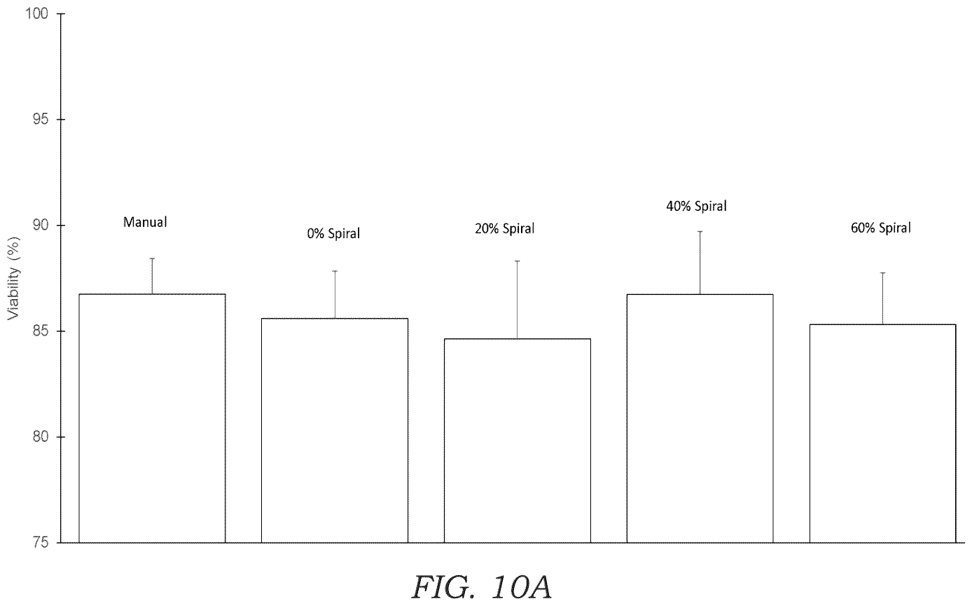

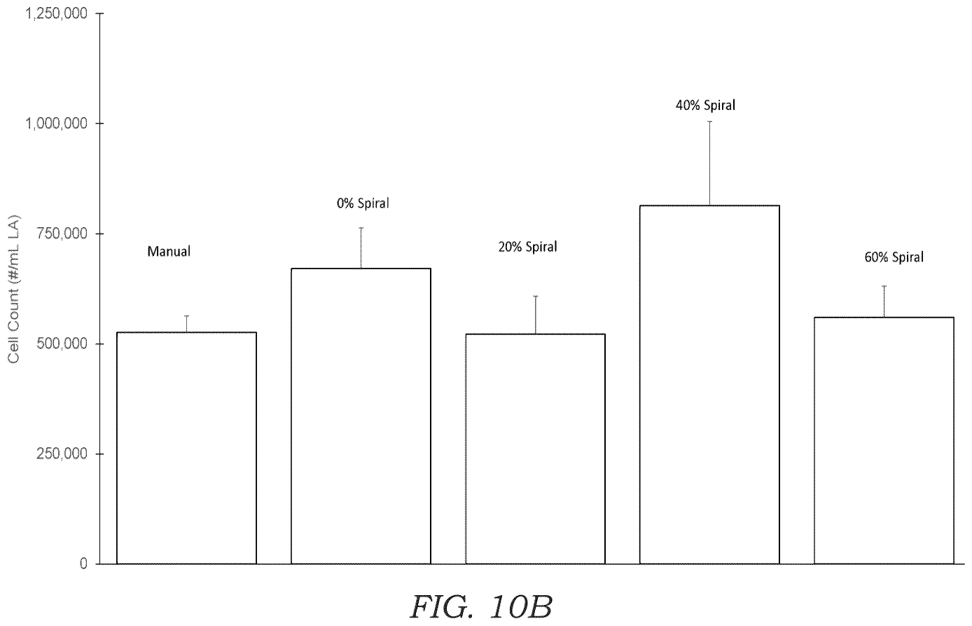

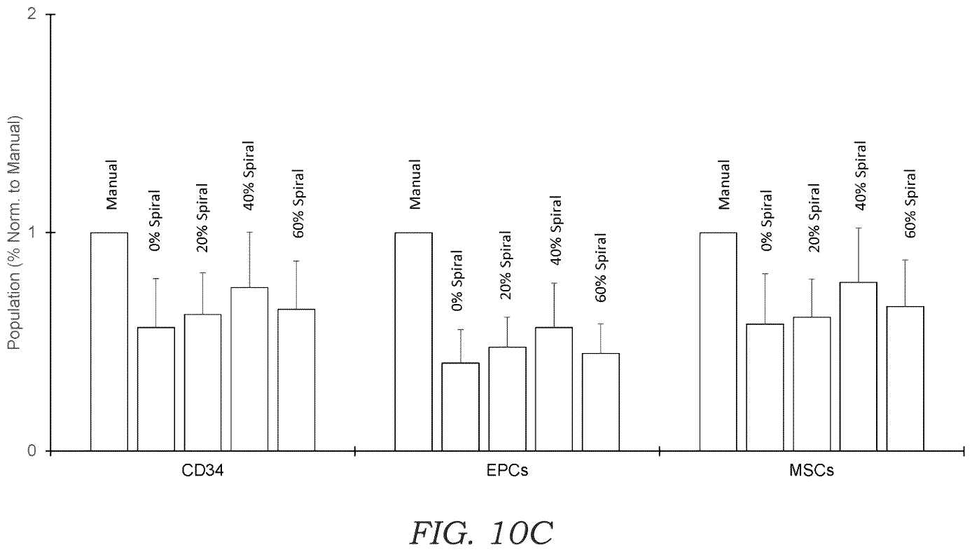

FIGS. 10A and 10B illustrate bar graphs of cell viability and cell count, respectively, in lipoaspirate 100 using manual washing, dynamic washing with 0% spiral ribs, 20% spiral ribs, 40% spiral ribs, and 60% spiral ribs. FIG. 10C illustrates bar graphs of various cell populations (CD34, EPCs, MSCs) obtained by processing lipoaspirate using manual washing, dynamic washing with 0% spiral ribs, 20% spiral ribs, 40% spiral ribs, and 60% spiral ribs.

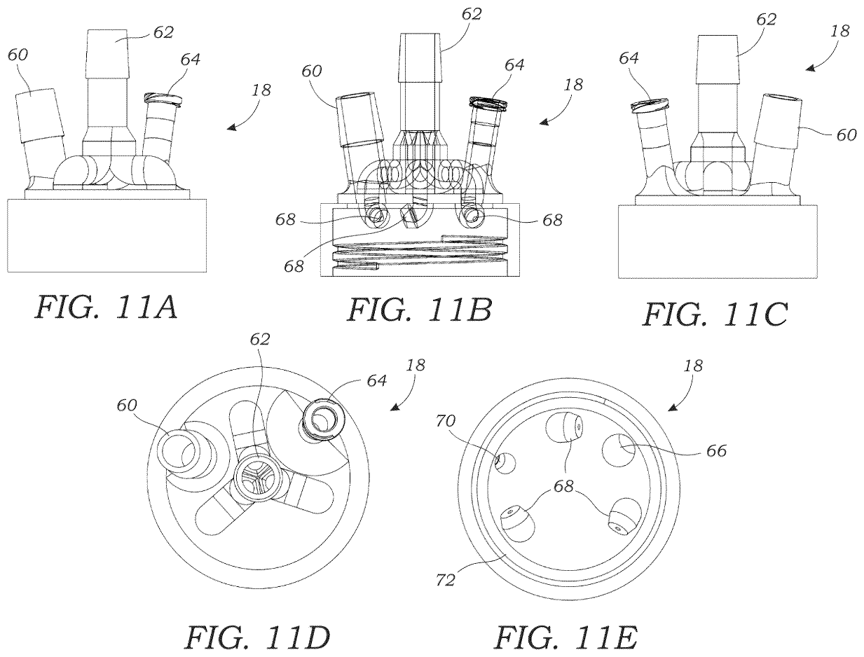

FIGS. 11A-11E illustrates various views of an alternative embodiment of the cap 18. In this embodiment, the cap 18 includes a lipoaspirate inlet 60, a saline inlet 62, and a vent inlet 64. With reference to FIG. 11E, the lipoaspirate inlet 60 leads to lipoaspirate outlet 66 that is directed to the lumen or opening 24 (FIG. 1A) of the washing device 10. The saline inlet 62 leads to a plurality (e.g., three) outlets 68 that are directed to the lumen or opening 24 of the washing device 10. The plurality of saline outlets 68 are configured as angled nozzles that direct flow toward the inner wall of the washing device 10. The vent inlet 64 leads to a vent outlet 70 that is directed to the lumen or opening 24 of the washing device 10. Both the lipoaspirate inlet 60 and the vent inlet 64 are positioned at an angle for easier connection to tubing or conduit 38. In addition, the angled configuration of the lipoaspirate inlet 60 helps direct flow to the center of the lumen or opening 24. A silicone gasket 72 is located inside the cap 18 and provides for sealing engagement with the cylindrical body 12 when screwed onto the threads 20 as seen in FIGS. 3A-3C. An advantage of this cap 18 is that it permits higher flow rates through the washing device 10 (e.g., 5 mL/second).

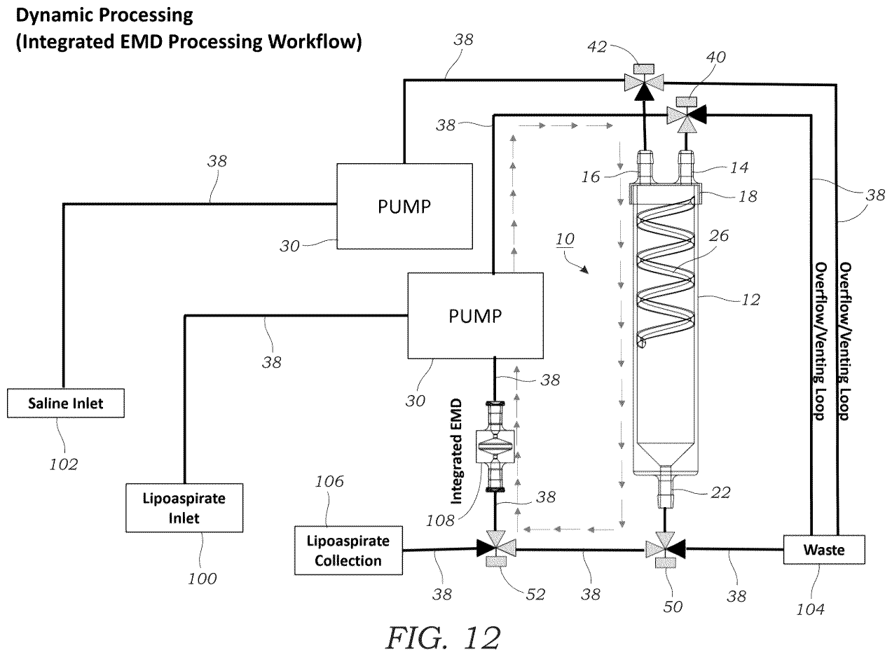

FIG. 12 illustrate another embodiment of the washing device 10 that integrates EMD 108 in a dynamic washing configuration. In this embodiment, two pumps 30 are provided with one pump 30 being used to pump lipoaspirate 100 and another pump 30 being used to pump saline 102. Alternatively, a single pump 30 may be used. For example, this may include a peristaltic pump with the ability to pump different amounts in different flow paths. The outlet 22 of the washing device 10 leads to a valve 50 that directs the contents to a waste receptacle 104 or to a second valve 52 that directs flow to either a lipoaspirate collector 106 or the EMD 108. Tubing or conduit 38 links the EMD 108 to the lipoaspirate pump 30 and returns flow back to the washing device 10 via the first inlet 14 or the second inlet 16.

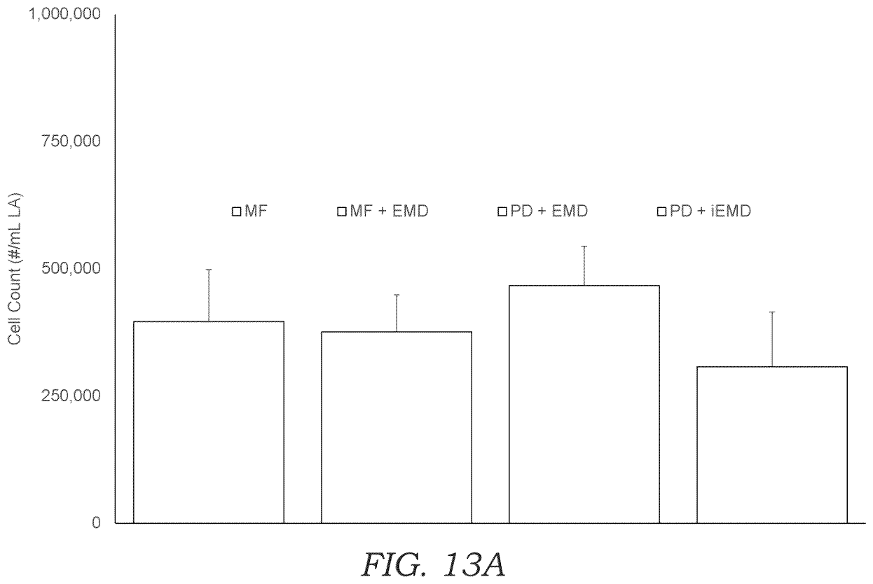

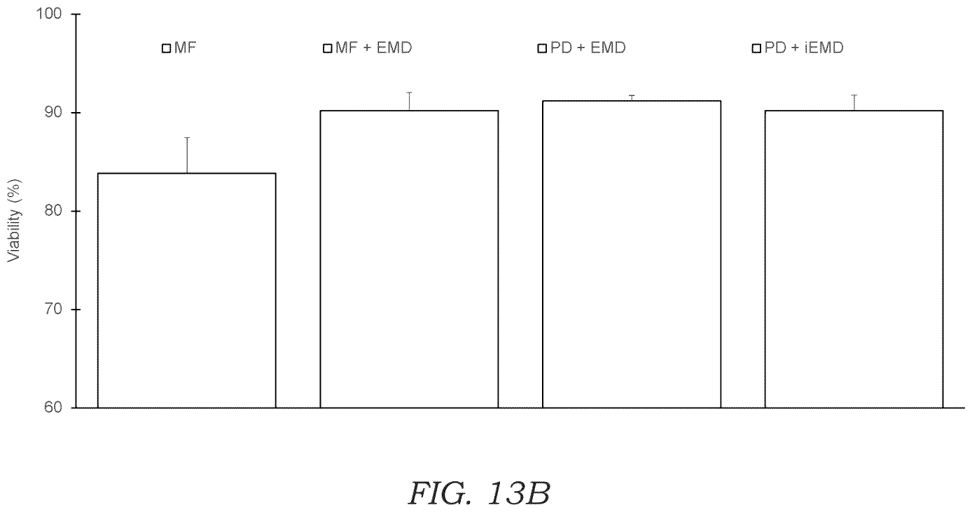

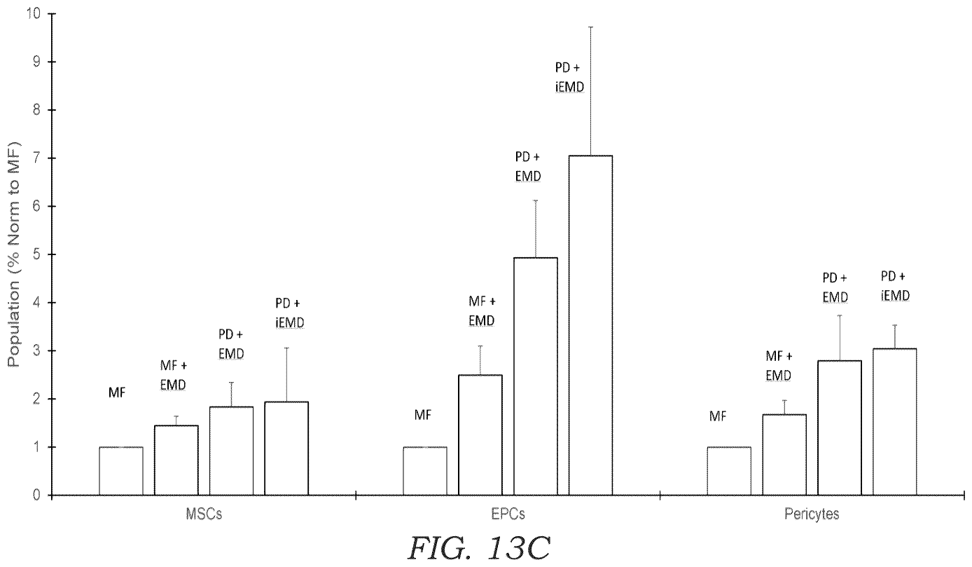

FIGS. 13A-13C illustrates data obtained using washing device 10 in the configuration of FIG. 12. Data obtained included cell counts (FIG. 13A), viability (FIG. 13B), and population change compared to manual washing (MF or macrofat) (FIG. 13C). Data obtained using the setup of FIG. 12 is labeled as iEMD while a syringe-based EMD setup that used a syringe pump+EMD with the washing device 10 is designated as EMD. MF indicates manually washed lipoaspirate. Cell counts were greater for lipoaspirate washed in the that underwent EMD processing, but overall, there were no considerable differences in both EMD processing modalities. Viability was ≥90% for EMD processed MF samples and both PD processed samples. Lipoaspirate 100 was examined for fold changes EPCs, MSCs, and pericyte cell populations. There was no considerable increase in MSC populations with EMD processing, however, there was a slight increase in pericyte populations with samples washed with the washing device 10. EPCs saw a large increase with both EMD and iEMD modalities, seeing the greatest increase with the iEMD configuration.

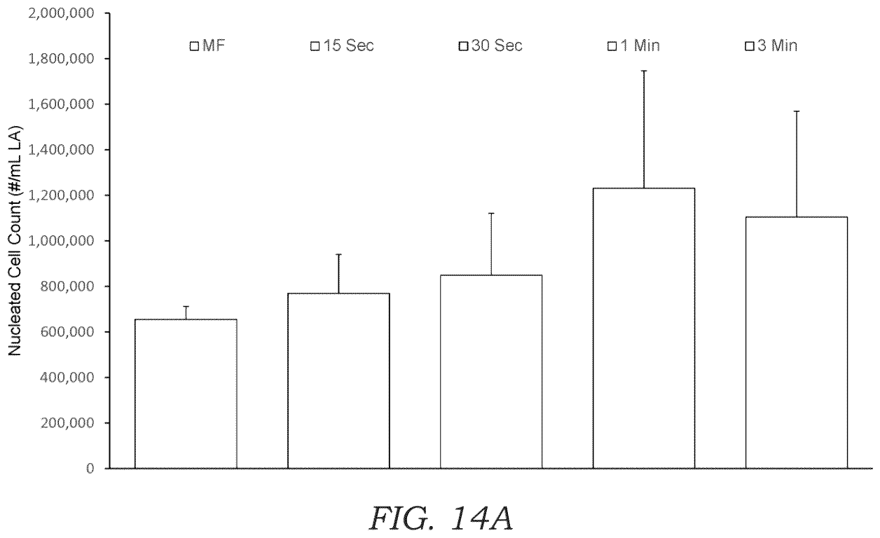

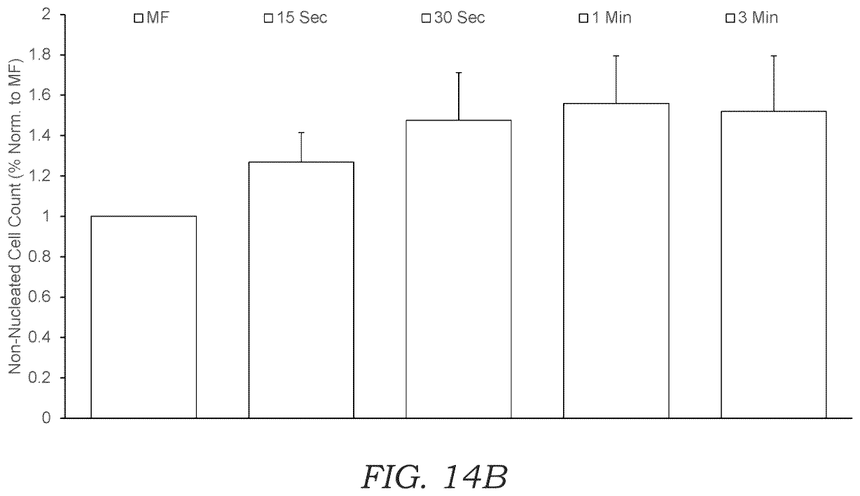

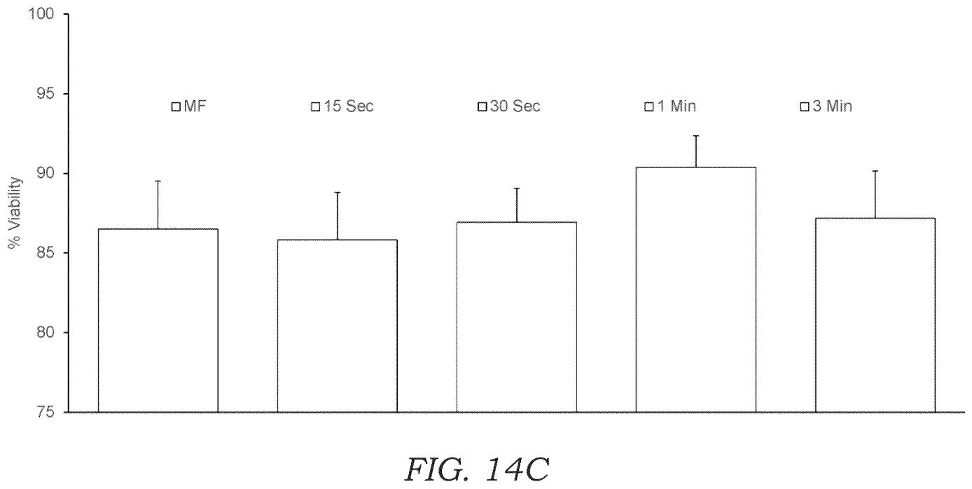

FIGS. 14A-14C illustrate the results of experiments done to optimize wash interval wait times using the setup of FIG. 12. A 5 mL/sec flow rate was used to optimize the wash protocol by exploring how varying loading durations between wash intervals affected the resultant lipoaspirate wash quality. Dynamic washing with the cap 18 of FIGS. 11A-11E and the washing device 10 were performed with loading at 15 mL/sec, followed by a 1-minute phase separation step prior to initiating washing. Washing was performed in 25 mL intervals and this data depicts the wait time between these 25 mL intervals. It was found that the 1-minute condition resulted in higher nucleated cell counts and viability. Non-nucleated cell counts were highest for the 1 minute, which meant that there was still a high degree of red blood cells in the lipoaspirate, however, the 1-minute condition offered minimal LA loss to the waste when compared to both 15 and 30 second conditions.

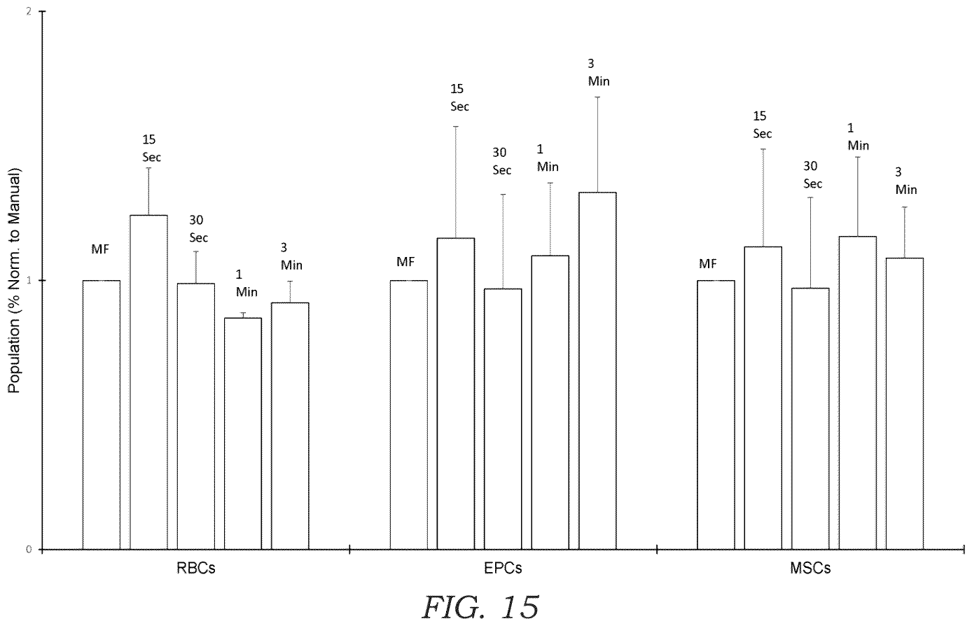

FIG. 15 illustrates cell population (% normalized to manual wash) for RBCs EPCs, and MSCs. Overall, a decrease in RBCs was found as interval wait time was increased. All washing conditions led to small changes in EPCs and MSCs, but none of the changes were statistically significant and overall showed equivalence to manual washing.

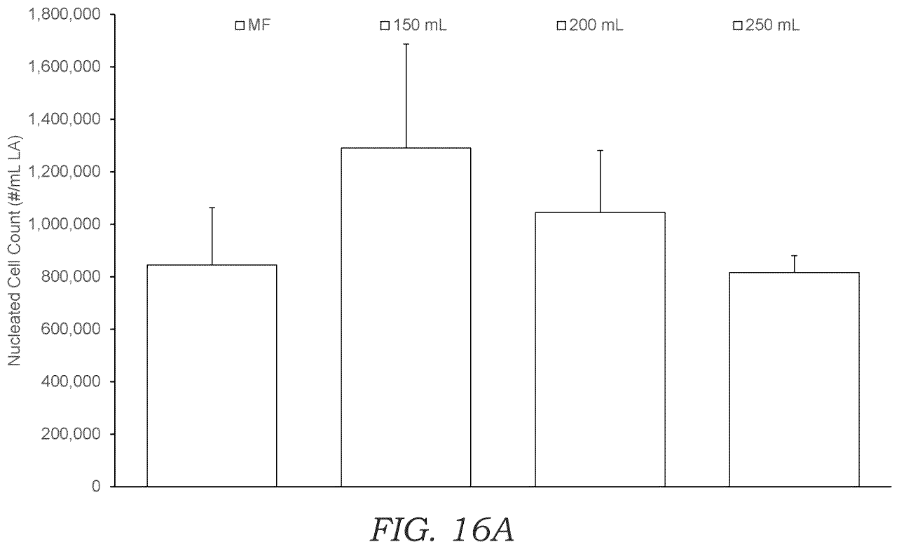

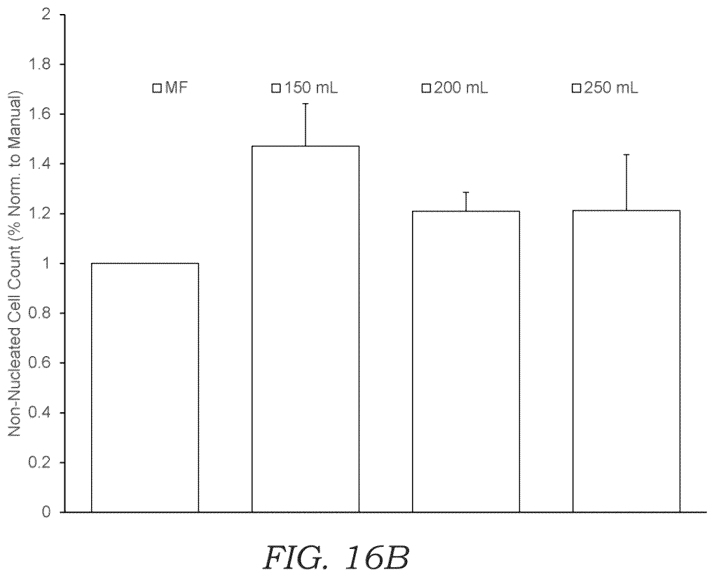

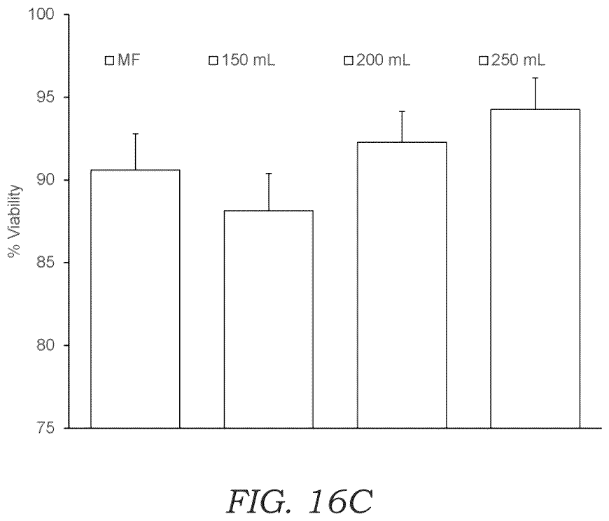

FIGS. 16A-16C illustrates the results of varying total wash volume on nucleated cell counts (FIG. 16A), non-nucleated cell counts (FIG. 16B), and viability (FIG. 16C). Using a 15 mL/sec loading flow rate, a 5 mL/sec saline washing flowrate and a 1-minute wash cycle wait time, various wash volumes were explored to further optimize the dynamic wash protocol. Looking at nucleated cell counts and viability, it appeared that 250 mL resulted in results closest to manual washing while having improved viability. Non-nucleated cell counts saw a decrease after 150 mL of wash volume, with no major differences between 200 and 250 mL.

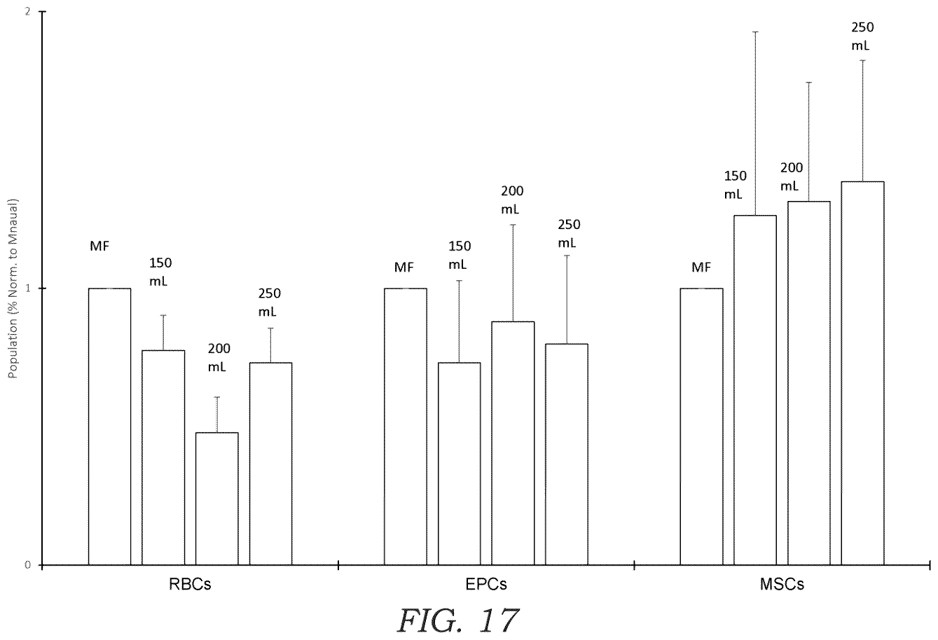

FIG. 17 illustrates the impact of total wash volume on RBC, EPC, and MSC subpopulations and showed that all RBC values were lower than manual washing. There was no considerable change when comparing all of these conditions, other than a slight overall increase in MSCs when compared to manual washing.

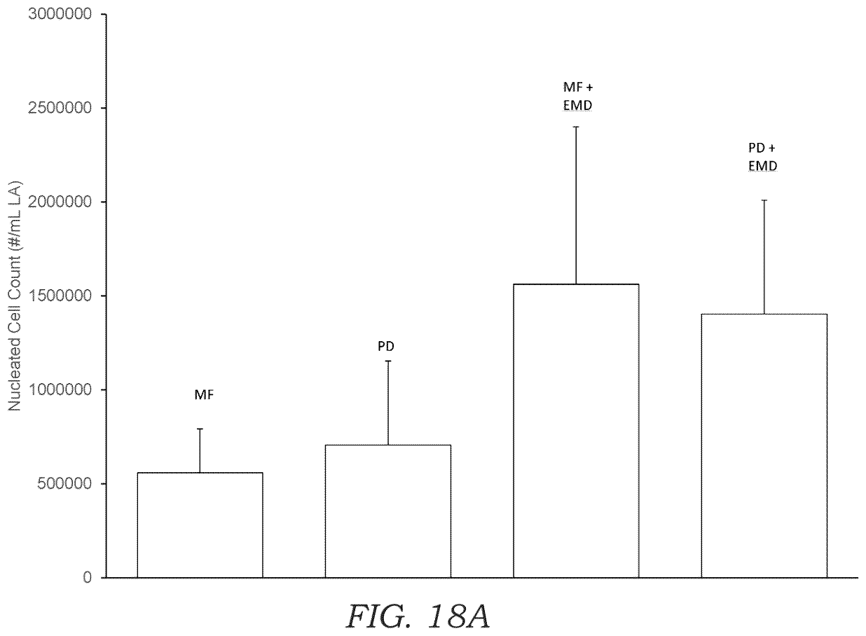

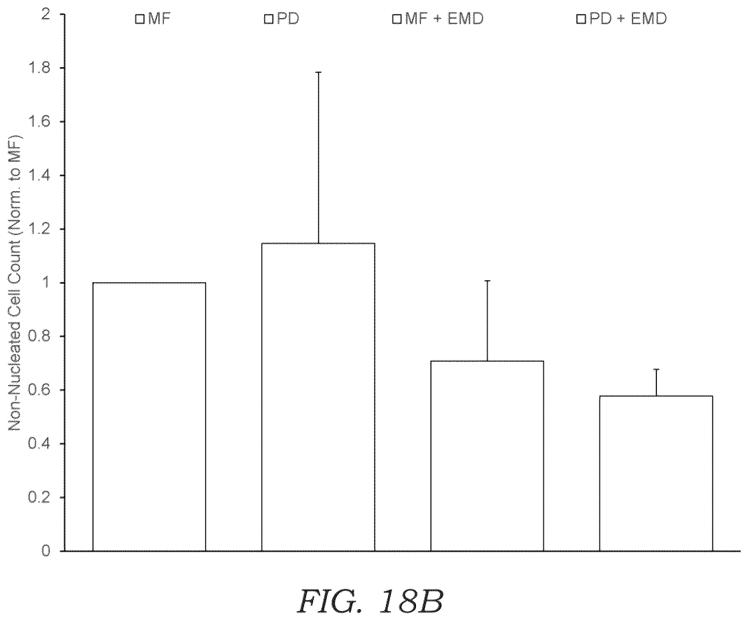

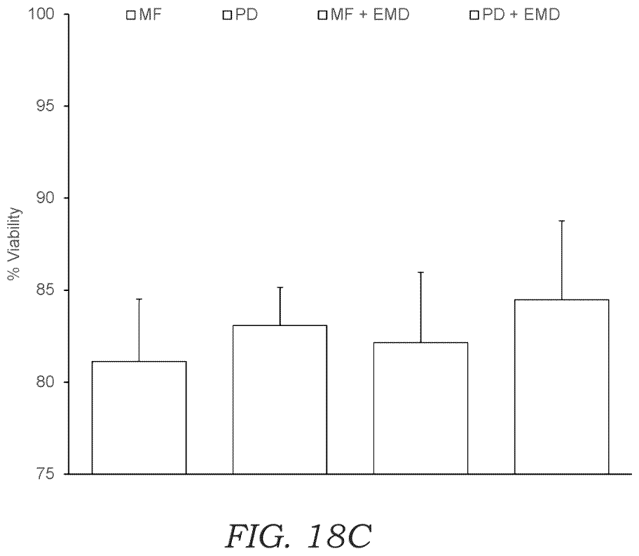

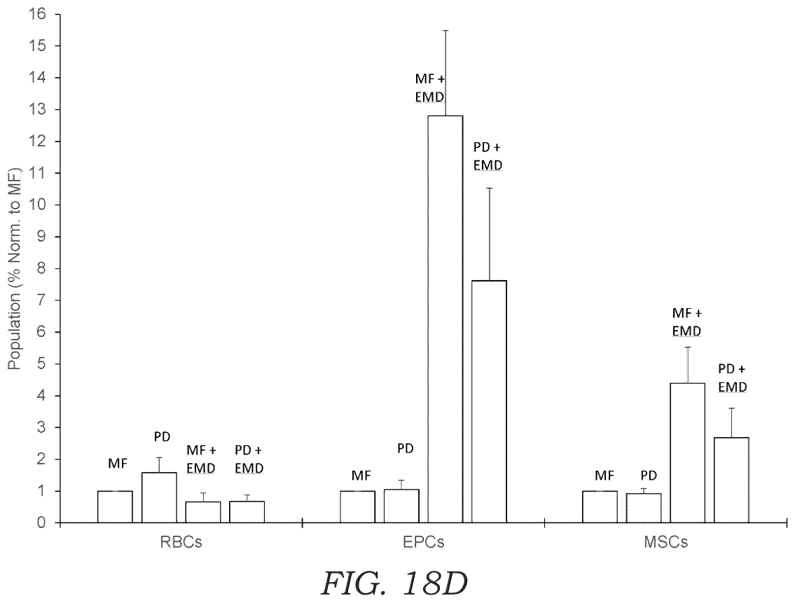

FIGS. 18A-18C illustrate results obtained using the washing device 10 with the cap 18 of FIGS. 11A-11E with and without a syringe-based EMD process. The results show that the washing device 10 does not adversely impact the lipoaspirate quality. As seen in FIG. 18A, while processing using the washing device 10 without EMD found slightly higher nucleated cell counts, after EMD processing, nucleated cell counts were similar and no major differences were seen. FIG. 18B illustrates the results for non-nucleated cell counts. The washing device 10 alone (PD) had slightly higher non-nucleated cell counts, however, after EMD processing, there was a slight relative drop compared to MF+EMD. Non-nucleated cell counts are used as a proxy or measure for RBCs. FIG. 18C illustrates that cell viability was above 80% for all processing conditions with no significant differences. FIG. 18D shows that RBCs were slightly higher than the MF control before EMD processing. However, after EMD processing both the MF and washing device 10 (PD) washed lipoaspirate samples 100 has lower quantities of RBCs. EPCs showed a significant increase after mechanical processing (EMD). MSCs showed an approximately 4-fold increase for the MF+EMD conditions and a 3-fold increase for the PD+EMD conditions).

A key advantage of the washing device 10 is that lipoaspirate 100 may be washed quickly and thoroughly from blood, drugs, and other waste products. In the dynamic operating mode, complete washing may be done in about 5 minutes or less. The lipoaspirate 100 that is washed with the washing device 10 may be directly used in fat grafting. For example, lipoaspirate may be harvested from a subject, washed using the washing device 10, collected and then injected as a fat graft back into the same subject. The washing device 10 may also be used in cellular therapeutics by combining the washing device 10 with the EMD 108 and/or filtration device 110 for regenerative medicine. In addition, the directly washed lipoaspirate 100 may be combined with a cellular therapeutic or supplement created with the EMD 108 and/or filtration device 110 to help the graft heal.

While the washing device 10 has particular application to fat grafts, the washed adipose tissue may also be subject to further downstream processing (e.g., emulsification and filtration) for increased activation of resident stem cell populations. These cells may then be used in treatments such as, for example, wound healing, multiple sclerosis, and Crohn's disease.

While embodiments of the present invention have been shown and described, various modifications may be made without departing from the scope of the present invention. The invention, therefore, should not be limited, except to the following claims, and their equivalents.

1. A washing device for washing of lipoaspirate comprising:
a cylindrical body having a first inlet and a second inlet located at one end thereof and an outlet located at opposing end of the cylindrical body; and
one or more spiral ribs disposed along an inner surface of the cylindrical body and extending from the end of the cylindrical body having the first inlet and the second inlet toward to the opposing end of the cylindrical body.

2. The washing device of claim 1, wherein the cylindrical body has a length and the one or more spiral ribs extend along substantially the entire length of the cylindrical body.

3. The washing device of claim 1, wherein the cylindrical body has a length and the one or more spiral ribs extend along ≤60% of the length of the cylindrical body.

4. The washing device of claim 1, wherein the cylindrical body has a length and the one or more spiral ribs extend along ≤40% of the length of the cylindrical body.

5. The washing device of claim 1, wherein the cylindrical body has a length and the one or more spiral ribs extend along ≤20% of the length of the cylindrical body.

6. The washing device of claim 1, further comprising one or more pumps fluidically connecting the first inlet to a source of lipoaspirate and connecting the second inlet to a source of buffer or saline solution.

7. The washing device of claim 1, further comprising a waste receptacle fluidically coupled to the outlet.

8. The washing device of claim 7, wherein a valve is interposed between the waste receptacle and the outlet, the valve further fluidically coupled to lipoaspirate collector, wherein actuation of the valve creates a flow path from the washing device to the waste receptacle or the lipoaspirate collector.

9. The washing device of claim 1, wherein the cylindrical body has a volume within the range of 10 mL to 1000 mL.

10. A method of washing lipoaspirate comprising:
providing a washing device having a cylindrical body having a first inlet and a second inlet located at one end thereof and an outlet located at opposing end of the cylindrical body, wherein one or more spiral ribs disposed along an inner surface of the cylindrical body and extending from the end of the cylindrical body having the first inlet and the second inlet toward to the opposing end of the cylindrical body;
loading the washing device with lipoaspirate and a buffer or saline solution via the first inlet and second inlet, respectively; and
continuously or semi-continuously pumping the buffer or saline solution into the washing device via the second inlet.

11. The method of claim 10, further comprising stopping pumping the buffer or saline solution into the washing device.

12. The method of claim 11, further comprising withdrawing washed lipoaspirate from the washing device via the outlet.

13. The method of claim 12, further comprising subjecting the washed lipoaspirate to further processing comprising applying shear forces on the washed lipoaspirate.

14. (canceled)

15. The method of claim 12, further comprising injecting the washed lipoaspirate as a fat graft into a subject.

16. The method of claim 15, wherein the washed lipoaspirate is combined with further processed lipoaspirate.

17. The method of claim 10, wherein the cylindrical body has a length and the one or more spiral ribs extend along ≤60% of the length of the cylindrical body.

18-19. (canceled)

20. A method of washing lipoaspirate comprising:
providing a washing device having a cylindrical body having a first inlet and a second inlet located at one end thereof and an outlet located at opposing end of the cylindrical body, wherein one or more spiral ribs disposed along an inner surface of the cylindrical body and extending from the end of the cylindrical body having the first inlet and the second inlet toward to the opposing end of the cylindrical body;
loading the washing device with a mixture of lipoaspirate and a buffer or saline solution via the first inlet and second inlet, respectively; and
recirculating the mixture of lipoaspirate and the buffer or saline solution from the washing device out of the outlet and into the first inlet or the second inlet.

21. The method of claim 20, further comprising stopping the recirculation of the mixture and allowing the contents of the mixture within the washing device to undergo phase separation.

22. The method of claim 21, further comprising removing waste eluent from the washing device via the outlet.

23. The method of claim 22, further comprising adding the buffer or saline solution via the second inlet and recirculating the mixture of lipoaspirate and the buffer or saline solution from the washing device for a period of time, stopping the recirculation to separate the washed lipoaspirate from waste eluent, and removing the waste eluent from the washing device via the outlet.
